(19) United States
(12) Patent Application Publication
(10) Pub. No.: US 2009/0192335 A1
(43) Pub. Date: Jul. 30, 2009

(54) TEMPERATURE-CONTROL IN THE PERFORMANCE OF OXIDATION REACTIONS OF HYDROCARBONS

(75) Inventors: Jobst Rüdiger von Watzdorf, Mannheim (DE); Gerhard Koppenhöfer, Römerberg (DE); Wolfgang Kanther, Dannstadt-Schauernheim (DE); Thomas Krug, Worms (DE); Thorsten Johann, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/301,184

(22) PCT Filed: May 11, 2007
(86) PCT No.: PCT/EP07/54555

§ 371 Date: Nov. 17, 2008

(30) Foreign Application Priority Data
May 17, 2006 (EP) 06114093.5

(51) Int. Cl. C07C 45/27; B01J 8/06

(52) U.S. Cl. 568/449; 422/188

(57) ABSTRACT

The present invention relates to a process for performing catalyzed oxidation reactions of hydrocarbons over a catalyst disposed in a reactor, which comprises cooling the product gas stream of the catalyzed oxidation reaction immediately after it leaves the reaction zone of the reactor by feeding in a temperature-controlled gas stream which is fed in through at least one feed device into the region between lower reactor plate and outlet of the product gas stream.

The present invention relates to a process for performing catalyzed oxidation reactions of hydrocarbons, especially to the preparation of vicinal dioxo compounds from the corresponding diols, and to a reactor comprising an apparatus for temperature adjustment in the outlet region of the reactor.

For processes which comprise a catalyzed oxidation reaction in the gas phase, for example the preparation of glyoxal, the use of tube bundle reactors is known from EP 1 169 119. In this case, the gas mixture is introduced into the reaction tubes in which a fixed bed of a catalytically active multimetal oxide is disposed. A heat exchange medium circuit is passed through the space which surrounds the reaction tubes between an uppermost and lowermost tube plate, in order to supply and to remove heat of reaction.

Known heat exchange media are temperature control media which are liquid within the range of the reaction temperatures existing; in particular melts of salts are used.

DE 1 923 048 discloses the preparation of glyoxal by oxidizing the hydroxyl compound ethylene glycol over a catalyst. Suitable oxidation catalysts mentioned are those which are obtained by oxidizing a copper-tin alloy, a copper-tin-phosphorus alloy or a copper-phosphorus alloy. The oxidation takes place in the gas phase, by reacting a hydroxyl compound, for example ethylene glycol converted to the gas phase in an evaporator, with an oxygenous gas, for example air, in the presence of the oxidation catalyst at elevated temperature. A diluent gas, for example nitrogen, carbon dioxide, steam or another gas which is inert toward the reaction participants and the products under the reaction conditions can be added to the gas mixture. In the case of preparation of glyoxal from ethylene glycol, typical reaction temperatures are from 300 to 450°0 C.

A disadvantage in the process described has been found to be that the yields of glyoxal achieved are comparatively low at from 65 to 70% based on the amount of ethylene glycol used.

Side reactions, especially in the downstream pipelines and apparatus, which may be either homogeneous gas phase reactions or heterogeneously catalyzed wall reactions, which lead to a yield loss of glyoxal, may be responsible for this. These unselective oxidation reactions take place to an increased extent at the high temperatures existing in the exit region of the glyoxal-comprising product gas stream in the reaction tubes.

U.S. Pat. No. 5,840,932 discloses, in a process for preparing ethylene oxide by catalytic oxidation in a tube bundle reactor, introducing a cooled substream prepared from the product stream into a region below the tubes and mixing it with the exiting product stream, with the aim of directly cooling the product stream in the exit region. The introduction is effected by means of a nozzle system not specified in detail.

DE 27 37 894 discloses intensively cooling a product gas stream in the preparation of maleic anhydride in a reaction vessel by bringing about heat exchange with a cooling liquid flowing in cooling coils or a temperature reduction by mixing with a cooling gas which may also be part of the reactor recycle gas, a part of an oxygenous gas or a part of the product stream. The cooling gas can be introduced via a gas distributor system, in the form of a sprinkler device, which is arranged in the lower region of the reaction vessel, i.e. in the reaction vessel hood. In this context, the high space demand of such a gas distributor system in the reaction vessel hood has a disadvantageous effect, as a result of which the costs of the reaction vessel can rise. Other problems have been found to be the devices flowed through by cold gas, on which condensation and/or de-sublimation processes can occur. The associated deposits on the devices can become detached from them and are entrained into downstream plant parts.

Using the example of the preparation of glyoxal, in spite of a high to virtually complete conversion of the monoethylene glycol reactant, it is found that the yields of glyoxal based on the monoethylene glycol used are low. In particular, the high temperatures which exist in the product gas stream induce a homogeneous and/or heterogeneous reaction of the glyoxal which can lead to significant yield losses in the lines and apparatus which follow downstream of the tube bundle reactor.

It is therefore an object of the present invention to provide a process concept and a corresponding apparatus which achieve improved yields in the performance of catalyzed oxidation reactions of hydrocarbons, especially in the preparation of vicinal dioxo compounds from corresponding diols, for example the preparation of glyoxal from the monoethylene glycol reactant.

The achievement of the object proceeds from a process for performing catalyzed oxidation reactions of hydrocarbons over a catalyst disposed in a reactor, such as the preparation of vicinal dioxo compounds from corresponding vicinal diols by catalyzed oxydehydrogenation reaction of the diols with atmospheric oxygen over a fixed catalyst bed arranged in the reaction tubes of a tube bundle reactor. It comprises cooling the product gas stream of the oxidation reaction immediately after it leaves the reaction zone of the reactor by feeding in a temperature-controlled gas stream.

Temperature control of a product gas stream immediately after it leaves the reaction zone can be applied at any point where a product which is thermally unstable under the existing conditions and can be stabilized by cooling with a temperature-controlled gas stream is present. The reaction zone comprises a region of a reactor in which a catalyst is disposed, for example, as a fluidized bed or as a fixed bed, especially in reaction tubes of a tube bundle reactor.

The process according to the invention will be described with reference to the preparation of vicinal dioxo compounds in a tube bundle reactor.

The process for preparing vicinal dioxo compounds comprises the provision of a reactant mixture which in particular comprises the corresponding vicinal diols. In the case of preparation of glyoxal by a catalyzed oxydehydrogenation of monoethylene glycol, the latter is converted to the gas phase in a suitable evaporator. The gaseous monoethylene glycol is admixed with a cycle gas which is inert toward the reaction participants under the existing reaction conditions and toward the products. The cycle gas may comprise essentially nitrogen and proportions of oxygen, carbon dioxide, carbon monoxide and water. For example, the cycle gas contains from 0 to 5% by volume of O₂, from 0 to 10% by volume of CO₂, from 0 to 5% by volume of CO, from 0 to 15% by volume of H₂O and an amount of nitrogen corresponding to the remainder. However, further constituents of the cycle gas are also possible. The oxygen required for the oxy-dehydrogenation of the vicinal diols can be provided by adding fresh air to the cycle gas saturated with vicinal diols.

The molar ratios of oxygen to ethylene glycol before entry into a tube bundle reactor are typically <1.5 mol of oxygen per mole of ethylene glycol.

The reactant gas stream prepared in this way is introduced into a reactor hood in a tube bundle reactor and passes into a multitude of reaction tubes comprising fixed catalyst bed. It is preferably a phosphorus-doped copper catalyst, but it is also possible to use silver catalysts which have been doped with gold, platinum, rhodium or palladium, silver catalysts comprising copper or catalysts based on molybdenum oxide.

The heat of reaction generated in the catalytic oxydehydrogenation is removed by a heat exchange medium surrounding the reaction tubes, preferably in the form of a salt circuit, and a system composed of vapor generator and vapor superheater. Thus, the heat of reaction removed can be utilized, for example, for the generation of process heat. Typically, the tube bundle reactor used has a one-zone configuration, but a multizone configuration with a plurality of reaction zones arranged in succession, in each of which different temperatures can be established by a separate heat exchange circuit, is also possible.

The tube bundle reactor used further comprises a cylindrical region which is typically concluded by hoods at both ends. In the cylindrical region, a multitude of reaction tubes is typically arranged between an uppermost and a lowermost tube plate. Typical diameters of the cylindrical region are from 2.5 to 5 m. Tube bundle reactors with such a diameter have generally from 1000 to 15 000 reaction tubes, preferably from 2000 to 10 000 reaction tubes. Typically, the internal diameter of the reaction tubes is from 20 to 70 mm, preferably from 40 to 60 mm. The typical length of the reaction tubes and hence the length of the cylindrical region of the reactor is in the range from 1.5 to 5 m, preferably from 2 to 3.5 m.

In the preparation of glyoxal, an entrance temperature in the salt circuit of from 360 to 390° C. has been found to be advantageous for the desired maximum conversion of the monoethylene glycol used over the catalyst. The temperature of the product gas stream leaving the reaction tubes is typically from 350 to 370° C. Subsequently, the product gas stream leaving the tube bundle reactor can be cooled in a cycle gas recuperator, and, in a subsequent quenching step, the condensable components which essentially contain glyoxal, and by-products such as formaldehyde, glycolaldehyde, formic acid and others, can be condensed out. There follow further workup steps which ultimately lead to purified glyoxal.

The process according to the invention envisages a temperature reduction of the product gas stream immediately in the exit region from the reaction zone. In the case of a tube bundle reactor, this is the region of the reactor hood below the exit orifice of the reaction tubes, so that undesired side reactions of the product can be suppressed very substantially, which are additionally responsible for a yield loss.

For this purpose, the invention provides a feed device in the exit region of the reactor, generally in the lower hood, especially immediately below the exit orifices of the reaction tubes in the case of a tube bundle reactor in the region between the lowermost tube plate and the outlet of the reactor. The inventive feed device enables feeding of a temperature-controlled gas stream which, by intensive mixing with the product gas stream, reduces the temperature.

The temperature-controlled gas stream fed in may be a cold, inert gas, preferably a substream of the cooled cycle gas. The gas stream is preferably fed in in a volume ratio of from 1:20 to 8:10 based on the product gas stream, especially in a ratio of from 1:10 to 1:5.

In order to achieve sufficient cooling of the product gas stream, which suppresses the undesired side reactions in a suitable manner, the temperature of the temperature-controlled gas stream fed in is generally from 30 to 300° C., preferably from 80 to 200° C. and more preferably from 80 to 120° C. In the cooling of the product gas stream, the integrated heat system should also be taken into account for the cycle gas preheating, so that there is a lower temperature limit depending on the process used. Using the example of the preparation of glyoxal, cooling of from 30 to 60° C. has been found to be suitable, the lower temperature limit being about 300° C.

In the process according to the invention, a substream of the cycle gas is fed in as an appropriately temperature-controlled gas stream. The product is removed from the cycle gas used in workup steps downstream of the catalytic oxidation reaction, and, after saturation with reactant and mixing with fresh air or reaction air, recycled back into the process with a suitable temperature. According to the invention, an appropriate substream is diverted from this cycle gas and fed into the region between lower reactor plate and outlet immediately below the exit orifices from the reaction zone, where it mixes with the product gas stream.

According to the invention, the temperature-controlled gas stream can be fed in radially or tangentially. Radially means that the temperature-controlled gas stream fed in flows out of the feed device into the interior of the reactor essentially at right angles to the reactor wall.

The temperature-controlled gas stream can be fed in via feed devices which are disposed in the wall of the inventive reactor and are configured, for example, as nozzles, which are preferably disposed in the region of the reactor wall between the lower reactor plate and the outlet of the reactor.

The temperature-controlled gas stream can be fed in radially or preferably tangentially at an angle relative to the radius by means of a plurality of feed devices distributed uniformly on the circumference of the wall of the reactor. The angles may be within a range of from 20 to 60°, preferably from 40 to 50°, relative to the radius of the reactor.

Tangential feeding preferably leads to flow of the temperature-controlled gas stream in circumferential direction along the inner wall of the reactor, preferably of the tube bundle reactor, in the region of the exit orifice from the reaction zone, preferably in the region of the exit orifices of the reaction tubes. In addition to a suppression of homogeneous gas phase reactions of the product as a result of the cooling, the effect is thus achieved that the product gas stream is displaced from the wall region, which prevents heterogeneously catalyzed reactions of the product at the wall.

In addition, the tangential feeding of the temperature-controlled gas stream offers the advantage that the reactor wall need not additionally be cooled, since energy release resulting from undesired subsequent reactions is prevented.

The effectiveness of the temperature control of the product gas stream obtained by the feeding depends upon rapid mixing of the gas streams. Appropriate selection of the feed devices used allows the achievement of high flow rates of the gas stream to be fed in, which allow rapid mixing and hence desired rapid cooling. The feed devices may in particular be configured as nozzles. In particular, a plurality of nozzles are arranged uniformly on the circumference, preferably from 4 to 8, preferentially 6 nozzles. The nozzles conclude flush with the inner wall of the hood. The nozzle cross section is selected such that an exit flow rate of the temperature-controlled gas stream which is in the range from 50 to 100 m/s can be achieved.

In an advantageous embodiment of the feed devices, a tangential feed direction of the temperature-controlled gas stream can be achieved, which allows the generation of a gas stream with a tangential flow component which generates a swirl running in circumferential direction, which achieves a further positive effect for rapid mixing through a turbulent flow state.

Caused by possible reactions and side reactions, the formation of deposits, especially in flow calming zones, can be promoted. In order that this can be largely prevented, it is advantageous to design the inner wall of the reactor smoothly in all of its regions, including the hoods. Accordingly, the feed devices are preferably configured so as to conclude flush with the inner wall of the reactor.

The invention will be illustrated in detail with reference to the drawing.

The drawings show:

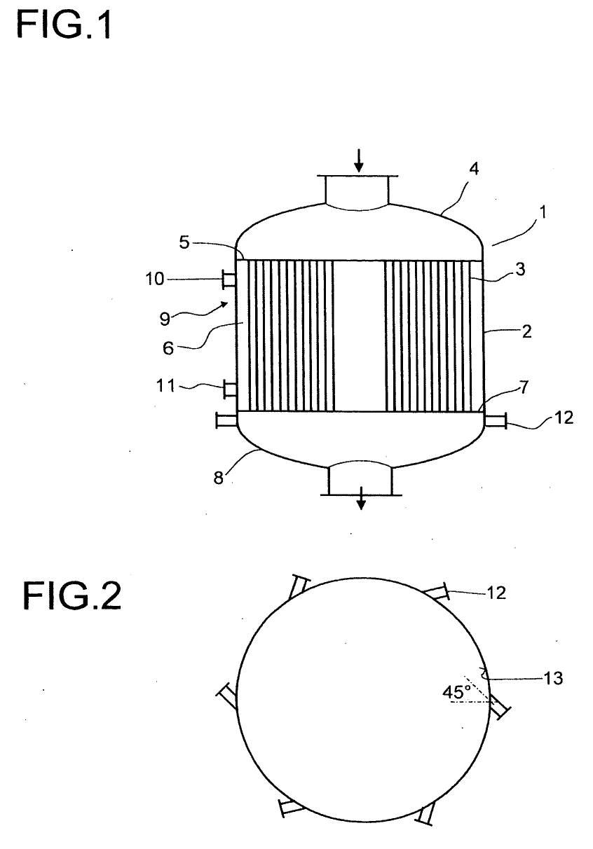

FIG. 1 an illustration of a tube bundle reactor used for the preparation of glyoxal in longitudinal section;

FIG. 2 an illustration of the tube bundle reactor from FIG. 1 in cross section.

FIG. 1 shows a schematic of a known tube bundle reactor 1 which comprises a cylindrical reactor jacket 2 in which the reaction tubes 3 are accommodated. A reactant mixture, which is composed of monoethylene glycol in the case of the preparation of glyoxal, air which is heated to a desired temperature, for example by a heater, and a cycle gas, passes into the tube bundle reactor 1, where it is distributed uniformly over the entire reactor cross section in the region of the upper hood 4. The upper hood 4 is concluded in the direction of the cylindrical reactor jacket 2 by an upper tube plate 5. The reaction tubes 3 of the reaction tube bundle 6 open into the tube plate 5. The reaction tubes 3, in their upper region, are welded with sealing to the tube plate 5. In the reaction tubes 3 is disposed the catalyst material (which is not shown). In their lower region, the reaction tubes 3 are welded with sealing to a lower tube plate 7 and open into a lower hood 8 of the tube bundle reactor 1. The monoethylene glycol-air mixture flows through the reaction tubes and is for the most part converted to glyoxal.

The reaction tube bundle 6 is temperature-controlled by a heat exchange medium circuit which is designated with reference numeral 9. For this purpose, for example, a salt melt is passed in and out through reactor jacket orifices 10, 11 in the cylindrical jacket section of the tube bundle reactor 1, and is passed there, in longitudinal flow, crossflow, countercurrent or cocurrent, past the reaction tubes 3 of the reaction tube bundle 6, in order to remove the heat of reaction formed in the oxydehydrogenation of monoethylene glycol.

The hot product gas stream is cooled directly in the lower hood 8 by feeding-in a temperature-controlled gas stream through feed devices 12 provided on the circumference.

The feed devices 12 are preferably arranged in one plane immediately below the lower tube plate 7 and hence immediately below the exit orifices of the reaction tubes.

FIG. 2 shows a cross section through the tube bundle reactor 1 of FIG. 1 in a plane in which the feed devices 12 are arranged. In a preferred embodiment, the feed devices 12 are arranged at an angle to the radius, for example at an angle of 45°. The feed devices 12 are distributed in a regular manner on the circumference of the lower hood 8, and conclude flush with an inner wall 13 of the tube bundle reactor 8.

The process according to the invention allows, by virtue of the corresponding temperature control of the product gas stream, the yield of the preparation of glyoxal to be increased by from about 3 to 5%. The space-saving arrangement of the feed devices immediately in the region of the exit of the product gas stream allows the costs of the reactor to be reduced, which are also influenced by the length of the hood of the reactor used. An advantageous alignment of the feed devices, which leads to a tangential flow component, can both bring about the intensive cooling of the product gas stream and also suppress catalyzed and unselective side reactions there by virtue of the type q flow generated along the inner wall.

1-16. (canceled)

17. A process for performing catalyzed oxidation reactions of hydrocarbons in a reactor comprising a catalyst containing reaction zone comprising a multitude of reaction tubes filled with catalyst between an upper and a lower tube plate, an inlet of an educt gas stream into an upper hood and an outlet for a product gas stream in a lower hood, wherein the product gas stream of the oxidation reaction is cooled immediately after it leaves the reaction zone of the reactor by radially and/or tan-gentially feeding in a temperature-controlled gas stream by means of at least one feed device provided on the reactor inner wall in the region between the loser tube plate and the outlet of the product gas stream.

18. The process according to claim 17, wherein the catalyzed oxidation reaction is an oxydehydrogenation reaction for preparing vicinal dioxo compounds from corresponding diols with atmospheric oxygen.

19. The process according to claim 17, wherein the reactor is a tube bundle reactor, the catalyst is disposed in the tubes of the tube bundle reactor and the temperature-controlled gas stream is fed in immediately below the exit orifices of the reaction tubes.

20. The process according to claim 17, wherein the temperature-controlled gas stream is a sub stream of a circulated cycle gas of the process.

21. The process according to claim 17, wherein the temperature of the temperature-controlled gas stream is in the range from 30 to 300° C.

22. The process according to claim 17, wherein the temperature of the temperature-controlled gas stream is in the range from 80 to 200°.

23. The process according to claim 17, wherein the temperature of the temperature-controlled gas stream is in the range from 80 to 120° C.

24. The process according to claim 17, wherein the temperature of the temperature controlled gas stream is fed into the product gas stream in a ratio of from 1:20 to 8:10.

25. The process according to claim 17, wherein the temperature of the temperature controlled gas stream is fed into the product gas stream in a ratio of from 1:10 to 1:15.

26. A reactor for performing catalyzed oxidation reactions by the process according to claim 17, comprising a reaction zone comprising a catalyst, composed of a multitude of reactor tubes filled with catalyst between an upper and a lower tube plate an inlet for a reactant gas stream into an upper reactor hood and an outlet for a product gas stream into a lower reactor hood, wherein at least one feed device for a temperature-controlled gas stream is provided on the inner wall of the reactor immediately after the product gas stream leaves the reaction zone in the region between the lower tube plate and the outlet of the product gas stream.

27. The reactor according to claim 26, wherein a multitude of reaction tubes which comprise a fixed catalyst bed and are arranged between an upper and a lower tube plate, and a heat exchange medium circuit are comprised in a space between upper and lower tube plate for removing heat of reaction, and wherein at least one feed device for a temperature-controlled gas stream is provided on the inner wall of the reactor immediately below the exit orifices of the product gas stream from the reaction tubes in the region between the lower tube plate and the outlet of the product mixture from the reactor.

28. The reactor according to claim 26, wherein the feed device is provided on the circumference of the inner wall of the reactor in the region of the reactor hood between the lower reactor plate and outlet of the product gas stream.

29. The reactor according to claim 26, wherein the feed device enables radial feeding of the temperature-controlled gas stream.

30. The reactor according to claim 26, wherein the feed device enables tangential feeding of the temperature-controlled gas stream at an angle relative to the radius from 20° to 60°.

31. The reactor according to claim 26, wherein the feed device enables tangential feeding of the temperature-controlled gas stream at an angle relative to the radius from 40° to 50°.

32. The reactor according to claim 26, wherein the feed device comprises one or more nozzles.

33. The reactor according to claim 32, wherein the nozzles conclude flush with the inner wall of the reactor.

34. The reactor according to claim 26, wherein the feed device feeds in the temperature-controlled gas stream with flow rates of from 50 to 100 m/s.
